(19) United States
(12) Patent Application Publication
(10) Pub. No.: US 2026/0301952 A1
(43) Pub. Date: Oct. 1, 2026

(54) PATIENT DIGITAL TWINS AND PATIENT BIOMIMETIC TWINS FOR PRECISION MEDICINE

(71) Applicant: University of Pittsburgh - Of the Commonwealth System of Higher Education, Pittsburgh, PA (US)

(72) Inventors: D. Lansing TAYLOR, Pittsburgh, PA (US); Alejandro Soto GUTIERREZ, Pittsburgh, PA (US); Albert H. GOUGH, Glenshaw, PA (US); Jaideep BEHARI, Sewickley, PA (US)

(73) Assignee: University of Pittsburgh - Of the Commonwealth System of Higher Education, Pittsburgh, PA (US)

(21) Appl. No.: 19/480,982

(22) PCT Filed: May 3, 2024
(86) PCT No.: PCT/US2024/027634

§ 371 Date: Nov. 3, 2025

Related U.S. Application Data
(60) Provisional application No. 63/464,106, filed on May 4, 2023.

(51) Int. Cl. G16H 50/20; G16H 20/17; G16H 50/70

(52) U.S. Cl. CPC G16H 50/20; G16H 20/17; G16H 50/70

(57) ABSTRACT

Embodiments described herein provide for a healthcare ecosystem of computing devices collecting disparate types (e.g., multimodal) of subject data and developing patient digital twins as predictive models configured to predict experimental outcomes for subjects. The system may inform clinicians of subject attributes for preparing microphysiology systems, sometimes referred to as “patient biomimetic twins” (PBTs), having attributes or disease derived from cells that can be used for experimentally testing the predictions generated by the predictive model of the PDT.

### CROSS-REFERENCE TO RELATED APPLICATIONS

This application claims the benefit of U.S. Provisional Application No. 63/464,106, filed May 4, 2023, which is incorporated by reference in its entirety.

### STATEMENT OF GOVERNMENT SUPPORT

This invention was made with government support under grant numbers DK117881, TR003289, DK119973, and TR004124 awarded by the National Institutes of Health. The government has certain rights in the invention.

### TECHNICAL FIELD

This application generally relates to developing and implementing patient biomimetic models and patient digital twins for predicting and validating proposed therapeutics.

### BACKGROUND

The FDA and other state and federal bodies impose strict regulatory science-based framework(s) governing efforts for modernizing, developing, evaluating, and deploying therapeutics (e.g., drugs) and therapeutic treatments for precision medicine. Conventionally, randomized controlled trials have been the gold standard for establishing efficacy and safety of new chemical entities for many decades. These trials, however, are expensive and often unwieldy to implement, especially for low-incidence diseases. Moreover, the high standards of scientific research observed by institutions, such as the FDA and universities, tend to skew research designs towards homogeneity, while not reflecting clinical real world inter-patient heterogeneity, which is a common cause for many late-stage development failures and poor response rates to drug candidates.

Thus, implementation of precision medicine, especially for complex and heterogeneous disease, faces many barriers. These barriers include, for example, selection of optimal subject cohorts for clinical trials for therapeutics, approval of therapeutics by regulatory agencies such as the FDA, and selection of patient-specific therapeutic strategies.

### SUMMARY

Disclosed herein are systems and methods capable of addressing the above-described shortcomings and may provide any number of additional or alternative benefits and advantages. Embodiments disclosed herein address challenges to implementing precision-medicine therapeutics (e.g., drugs, treatments). Embodiments described herein provide for a healthcare ecosystem of computing devices for hosting and implementing a system for therapeutics services. As explained herein, the computing devices integrate and extend several technologies for various functions for addressing shortcomings in the art, such as: creating subject (e.g., individuals participating in a study, medical patients) study groups in clinics and collecting disparate types (e.g., multimodal) subject data from various data sources; developing data-centric representations of patients, such as patient digital twins (PDTs), ingested by machine-learning models trained to predict patient-specific disease progression and responses to therapeutics; developing patient-specific microphysiology systems, sometimes referred to as “patient biomimetic twins” (PBTs), of disease derived from patient cells that can be used for experimentally testing predictions made by the machine-learning model using the PDT; and (d) implementation of an extendable database and analytics platform designed to access, manage, analyze, selectively share and computationally model patient-specific PDTs and PBTs data.

In an embodiment, a computer-implemented method comprises obtaining, by a computer, subject data for a subject from one or more subject databases, the subject data including a plurality of types of subject data; generating, by the computer, a predicted output by applying a predictive model of a patient digital twin on the subject data for the subject, the patient digital twin including the predictive model configured to generate a first predicted outcome of a testing configuration; updating, by the computer, a parameter of the predictive model according to a user input; generating, by the computer, a second predicted outcome by applying the patient digital twin on the subject data and the testing configuration, wherein the patient digital twin for the subject contains biomimetic twin configuration data associated with a patient biomimetic twin for the subject, and wherein the patient biomimetic twin includes an experimental representation of the subject according to the biomimetic twin configuration data; obtaining, by the computer, experimental result data for the subject associated with the patient biomimetic twin for the subject and the testing configuration; and updating, by the computer, the parameter of the patient digital twin, by applying the model of the patient digital twin on the subject data and the experimental result data for the subject. The computer may update one or more parameters the patient digital twin by, for example, applying one or more types of computational models (e.g., machine-learning, mechanistic, stochastic, and Bayesian networks) of the patient digital twin on the subject data and the experimental result data for the subject.

In another embodiment, a system comprises a computer comprising at least one processor and configured to obtain subject data for a subject from one or more subject databases, the subject data including a plurality of types of subject data; generate a predicted output by applying a model of a patient digital twin on subject data for the subject, the patient digital twin configured to generate a first predicted outcome of a testing configuration; update a parameter of the model according to a user input; generate a second predicted outcome by applying the patient digital twin on the subject data and the testing configuration, wherein the patient digital twin for the subject contains biomimetic twin configuration data associated with a patient biomimetic twin for the subject, and wherein the patient biomimetic twin includes an experimental representation of the subject according to the biomimetic twin configuration data; and obtain experimental result data for the subject associated with the patient biomimetic twin for the subject and the testing configuration; and update the parameter of the patient digital twin to train the patient digital twin, by applying a variety of computational models including machine-learning, mechanistic, stochastic, and Bayesian networks of the patient digital twin on the subject data and the experimental result data for the subject.

In another embodiment, a method for developing and administering precision treatments comprising obtaining subject data and a patient digital twin for a subject according to according to one or more treatment configurations indicating a treatment; obtaining a predicted treatment outcome for the treatment according to a predictive model of the patient digital twin; constructing a patient biomimetic twin for the subject based upon biomimetic twin configuration data indicated by the subject data for the subject; obtaining experimental result data by subjecting the patient biomimetic twin to the treatment; entering the experimental result data into a computer, wherein the computer updates the predictive model of the patient digital twin based upon a level of error between the experimental result data and the predicted treatment outcome; and administering the treatment to the subject.

In another embodiment, a method for identifying subject cohorts comprising obtaining subject data of a plurality of subjects, the subject data of each subject including one or more attributes of the subject; obtaining a cohort of subjects for a treatment trial according to one or more treatment configurations, wherein a computer applies a cohort prediction engine on the subject data of the plurality of subjects using the one or more treatment configurations; for each subject of the cohort, constructing a patient biomimetic twin for the subject based upon biomimetic twin configuration data indicated by the subject data for the subject; obtaining the experimental result data associated with the patient biomimetic twin of the subject from a laboratory testing device by subjecting the patient biomimetic twin to the treatment for the treatment trial; and administering the treatment to the cohort of subjects.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 shows components of a system for therapeutics modeling using a machine-learning architecture for processing various types of subject data, according to an embodiment.

FIG. 2 shows data flow among devices of a system for PBT development and experimentation using predictive PDTs, according to an embodiment.

FIG. 3 shows operations of a method for developing PDTs for predicting and using PBTs in experimental analysis, according to an embodiment.

FIG. 4 shows data flow among devices of a system for developing and using predictive PDTs and PBTs for experimentation, according to an embodiment.

### DETAILED DESCRIPTION

Reference will now be made to the illustrative embodiments illustrated in the drawings and claims, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

Described herein are systems and methods for developing patient digital twins (PDTs) as computational models representing patients or subjects from disparate subject data sources, where the PDT include predictive machine-learning models trained to predict therapeutic outcomes on the biology of the subject or cohort of subjects. The PDT may include trained, or tuned, hyper-parameters or weights in the various layers a machine-learning architecture. The layers of the machine-learning model of the PDT may implement any number of machine-learning techniques, artificial intelligence techniques, and handcrafted mechanistic techniques, among others. A computing device may apply the machine-learning architecture of the PDT on the subject data to predict outcomes of proposed therapeutics (e.g., drugs, clinical treatments). It should be appreciated that the terms machine-learning and artificial intelligence are intended referring to similar data-driven inferential functions or features, and not intended to be exclusive of one another. References to machine-learning functions are not intended to be exclusive of artificial intelligence functions.

Embodiments may implement various handcrafted models for generating and developing the PDTs. The computing device develops the handcrafted models using prior knowledge and expertise of the therapeutics testing scenario being modeled. A clinician-user may submit or enter user inputs to a user interface that, over time, calibrates various parameters or weights of the handcrafted models.

As an example, the handcrafted models may include mechanistic models. The mechanistic models include algorithmic functions to represent or model interactions between different components of clinical research. The goal of a mechanistic model is to explain how the components interact with one another, and/or predict how the components will behave under desired testing conditions, among other benefits. Unlike machine learning models or artificial intelligence models, which rely on statistical patterns in data to generate predictions, mechanistic models are based on fundamental principles and do not require large amounts of training data. The mechanistic models may be employed in clinical research, where the mechanistic models may be used to, for example, model the effects of therapeutics (e.g., drugs or treatments) on biological systems, predict the outcomes of clinical trials, and optimize treatment plans for individual patients. The mechanistic models may be implemented to model and study progression of diseases and identify and model new drug targets.

As another example, the computing device may implement a handcrafted stochastic model. Stochastic models may be handcrafted models or machine learning models. Handcrafted stochastic models are developed using prior knowledge and expertise of the therapeutic test scenario being modeled, and typically involve specifying a set of probability distributions and algorithmic functions that govern the behavior of the interacting components. Machine learning stochastic models are developed using data-driven approaches, where the computing device identifies statistical patterns and correlations used to output predictions of modeling a given set of inputs for the therapeutic test scenario.

The computing device may also output information that informs clinicians on creating patient biomimetic twins (PBT) as physical testing targets, sometimes called “organs-on-chips” (OoCs), corresponding to the PDTs. The computing device may execute machine-learning and artificial intelligence programming that derives causal inferences from experimental results data from experimental analysis (e.g., experiments) performed using the subject PBTs. The use of finely-tuned PBTs, made possible by continually tuned predictive PDTs, may beneficially overcome the limitations of and reduces the reliance on animal models.

The PBT (sometimes referred to as microphysiology system (MPS) or biomedical model) serves as a subject experimental representation used to understand normal and abnormal functions and provide a basis for preventive or therapeutic intervention in human disease. In some embodiments, the subject experimental representation may include 3D-layered cells. The 3D layered cells are produced by a combination of sequential cell layering and cell-to-cell self-organization of specific cell types. The cell types migrate and self-assemble into distinct layers forming tissue structures.

The combination of the computational models (the PDTs) and subject experimental models (the PBTs) described herein beneficially provides an efficient and safer understanding of, for example, disease mechanisms, characterization of potential drug activity and toxicity (e.g., dosing predictions and mechanisms of action), and identification of potential clinical biomarkers at various levels of people (e.g., population level, specific cohort subgroups, or individual subjects). Embodiments further enables prediction and testing of drugs for consequences of chronic exposure and the effect of drugs on specific cohort populations that would be otherwise unachievable before administering to the subjects (in real life).

As used herein, the terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human. As used herein, the terms “treatment” or “treating” as used herein with reference to reducing or eliminating a disease and/or improving or ameliorating one or more symptoms of a disease.

### Example System Components

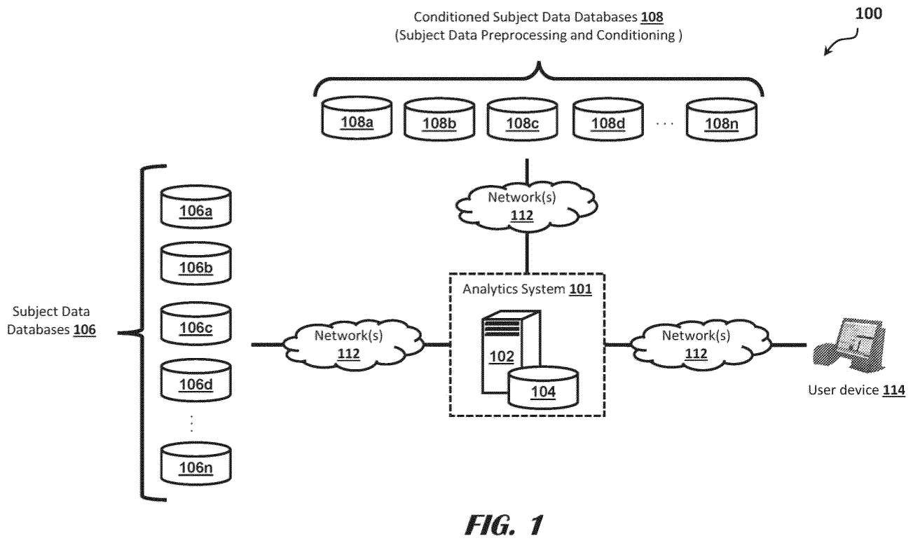

FIG. 1 shows components of a system 100 for therapeutics modeling using a machine-learning architecture for processing various types of subject data, according to an example embodiment. The system 100 includes a therapeutics analytics system 101 having analytics servers 102 and analytics databases 104 (sometimes referred to as a “federated database”), subject data databases 106a-106n (generally referred to as subject databases 106 or a subject database 106), conditioned subject data databases 108a-108n (generally referred to as conditioned databases 108 or a conditioned database 108), and user devices 114. One or more networks 112 interconnect the components of the system 100, allowing the devices to communicate with one another.

Embodiments may comprise additional or alternative components or omit certain components from those of FIG. 1 and still fall within the scope of this disclosure. It may be common, for example, to include multiple analytics servers 102. Embodiments may include or otherwise implement any number of devices capable of performing the various features and tasks described herein. For instance, FIG. 1 shows the analytics server 102 as a distinct computing device from the analytics database 104. In some embodiments, the analytics database 104 includes an integrated analytics server 102.

A therapeutics modeling service may host, implement, operate, or otherwise administer the analytics system 101 to provide various benefits associated with the therapeutics modeling functions and features described herein. Non-limiting examples of therapeutic modeling include predicting and evaluating drug efficacy, ADME-Tox, clinical trials, and therapeutic strategies. The analytics system 101 may execute the PDT to assess the subject's attributes to predict, for example, subject-specific mechanisms of biological responses to the testing configurations, such as predicting disease progression and/or therapeutic strategies. As an example, the analytics system 101 may predict a cohort for testing a drug or treatment using PDTs representing the subjects. As another example, the analytics system 101 may predict a drug or treatment efficacy for a particular subject (e.g., patient) using the PDTs respecting the subjects.

A clinician-user interacting with the analytics system 101 using the user device 114 may reference the PBTs logically corresponding to the PDTs. The PBTs include physical testing biological material created by the clinician using samples that the clinician (or other actor) extracted from the subjects represented by the corresponding PDTs. The clinician prepares and conducts experiments on the PBTs to test or validate the predictions generated by the PDTs (e.g., predicted therapeutic strategies). In some cases, the PDTs may be manually optimized according to inputs from users by operating the user device 114 to submit updates to the PDTs. Additionally or alternatively, in some cases, the PDTs may be algorithmically optimized by the analytics server 102 according to various machine-learning techniques, as implemented by the PDTs and/or the analytics server 102 using experimental result data.

The system 100 includes one or more networks 112, which may include any number of internal networks, external networks, private networks (e.g., intranets, VPNs), and public networks (e.g., Internet). The networks 112 comprise various hardware and software components for hosting and conduct communications amongst the components of the system 100. Non-limiting examples of such internal or external networks may include a Local Area Network (LAN), Wireless Local Area Network (WLAN), Metropolitan Area Network (MAN), Wide Area Network (WAN), and the Internet. The communication over the networks 112 may be performed in accordance with various communication protocols, such as Transmission Control Protocol and Internet Protocol (TCP/IP), User Datagram Protocol (UDP), and IEEE communication protocols, among others.

In some cases, the analytics system 101 may include a computing network infrastructure comprising physically and logically related software and electronic devices, managed or operated by, for example, a therapeutic modeling service provider, where the devices of the infrastructure 101 are configured to provide the intended therapeutic modeling services. The analytics system 101 may include an internal network (not shown) comprising the networking hardware and software components for hosting and conducting the communications amongst components of the analytics system 101, including communications between the analytics server 102 and the analytics database 104.

The user device 114 allows the user (e.g., clinician, medical provider, researcher) to interact with the therapeutic modeling services of the analytics system analytics system 101. The user device 114 may include any computing device comprising hardware (e.g., non-transitory machine-readable storage media, processors) and machine hardware-executed software components capable of performing the processes and tasks described herein. Non-limiting examples of the user device 114 may include a personal computer (PC) (workstation computer, laptop), tablet, and smartphone, among other types of electronic devices capable of performing the functions of the user device 114 described herein.

The user device 114 comprises, or couples to, peripheral devices for receiving the user inputs, such as user I/O devices (e.g., keyboard, mouse, monitor), allowing the user to interact with the user device 114 and the analytics system 101, via the network 112. As an example, the user may enter user inputs indicating experimental testing configurations, experimental testing results, PDT configurations, and PDT updates, among other types of inputs. In some cases, the peripheral devices may include instruments that obtain (e.g., receive, generate) the experimental results data when conducting experimental analysis on the PBT, and connectedly or wirelessly transmit the experimental results data to the user device 114 or to the analytics system 102. Non-limiting examples of such instruments include an image reader, microscope, live cell image reader, sequencing system, thermal cycler, and gel and blot readers. Additionally or alternatively, the user may enter the experimental results data into a user interface of the user device 114, which may in turn transmit the experimental results data to the analytics system 101 via the network 112.

The user device 114 executes various software programming for accessing the analytics system 101 via the one or more networks 112, allowing the user to provide user inputs to the analytics system 101 and receive outputs and data returned from the analytics system 101. In some implementations, the user device 114 executes locally installed software associated with the analytics system 101 for accessing and interacting with the services of the analytics system 101, and performing the various functions and features described herein. In some implementations, the user device 114 executes a web browser programing that accesses a website or web-app hosted by a webserver program executed by the analytics server 102 of the analytics system 101. The user may operate the web browser as the user interface for interacting with the services of the analytics system 101 and performing the various functions and features described herein.

The user may operate the user device 114 to submit requests for the analytics server 102 to perform the therapeutics services, such as requests for predicted cohorts or requests for predicted therapeutics efficacy. The requests include machine-readable instructions that instruct the analytics server 102 to perform the requested activity, such as determine cohort of subjects for testing a proposed therapeutic or determine a therapeutics result for a proposed therapeutic, among other potential processes. The user device 114 receives the user inputs indicating the request for the particular process and various configuration inputs associated with the particular request, and transmits the request to the analytics system 101 via the network 112.

The system 100 includes any number of subject databases 106 containing various types of subject data. A subject database 106 may be hosted on one or more computing devices comprising hardware components (e.g., non-transitory machine-readable storage media, processor) and software components (e.g., database management system (DBMS)) capable of performing the various tasks and processes described herein. Similarly, a computing device may host one or more subject databases 106. In some cases, certain subject databases 106 may be a component of the analytics system 101. Additionally or alternatively, in some cases, certain subject databases 106 may be outside of the analytics system 101, and hosted by another enterprise infrastructure network, such that the analytics server 102 (or other component of the analytics system 101) ingests the subject data from the subject database 106 via the network 112.

The subject databases 106 include various types of data used for generating or updating PDTs. Non-limiting examples of the subject databases 106 include clinical recordings database 106a, omics data 106b, MRI imaging 106c, and mobile health data 106d.

A clinical database 106a may include demographics data indicating the subject's age, gender, race, and ethnicity. The clinical database 106a may include medical history and comorbidities data indicating a subject's disease history and surgical history. The clinical database 106a may include body composition data indicating a subject's height, weight, fat mass, and the like. The clinical database 106a may include medications data indicating a subjects current and past medications. The clinical database 106a may include biochemistries data to indicate a subject's blood chemistry. The clinical database 106a may include patient reported outcome measures to indicate a subject's self-reported depression, anxiety, social function, or the like. The clinical database 106a may include noninvasive liver fibrosis staging to indicate a subject's liver stiffness measurements and controlled attenuation parameters. The clinical database 106a may include liver biopsy data to indicate a subject's steatosis grade score, inflammation score, ballooning score, and fibrosis stage. The clinical database 106a may include salivary and stool data to indicate a subject's taxonomic classification and species abundance. The clinical database 106a may include plasma and serum data to indicate a subject's metabolite and proteomic profiles. The clinical database 106a may include genetics data to indicate a subject's exome sequencing profile. Non-limiting examples of data in a clinical database 106a (or other subject databases 106) is shown and described in TABLE 1.

TABLE 1
Example Subject Clinomics Data Being Collected

| Clinical Data Domains | Measurement Methods | Variables |
|---|---|---|
| Demographics | Electronic health records | Age, gender, race/ethnicity |
| Morphometrics | Clinical scale, measuring | Height, weight, waist circumference, |
| | tape | BMI (calculated), waist/height ratio |
| | | (WHR) |
| Medical History & | Electronic health records | Medical: Diabetes, hypertension, |
| comorbidities | | dyslipidemia, coronary artery disease, |
| | | obesity, hypothyroidism, sleep apnea, |
| | | polycystic ovary, hypogonadism, |
| | | hypopituitarism, gout, cholelithiasis, |
| | | cancer. |
| | | Surgical: Cholecystectomy, weight |
| | | loss surgery, appendectomy, |
| | | tonsillectomy, bowel resection |
| Body composition | InBody body impedance | Height, weight, fat mass, lean mass, |
| | analyzer. MRI scan | total body water. Automated imaged |
| | | analysis for visceral and subcutaneous |
| | | adipose tissue (MRI - see below) |
| Medications | Electronic health records | Medications for diabetes, hypertension, |
| | | dyslipidemia, weight loss, NASH, heart |
| | | disease, steroids, gout. |
| Biochemistries | Electronic health records | Hemoglobin, platelet count, ALT, |
| | | AST, Total bilirubin, albumin, alkaline |
| | | phosphatase, GGT, PT/INR, total |
| | | cholesterol, LDL-C, HDL-C, |
| | | triglyceride, BUN/creatinine |
| Patient Reported | Promis-29 instrument | 7 domain scores (depression, anxiety, |
| Outcome measures | | physical function, social function, |
| | | fatigue, pain interference and sleep) |
| Nutrition and Lifestyle | National Cancer Institute | 65 nutrients and 37 food groups |
| Questionnaires | ASA24 questionnaire | |
| Noninvasive liver | Transient Elastography | Liver stiffness measurement. |
| fibrosis staging | | Controlled attenuation parameter |
| Cross-sectional | MRI abdomen with MR | Liver fat fraction. Liver stiffness score. |
| abdominal imaging | elastography and proton | Presence of portal hypertension. |
| | density fat fraction | Presence of cirrhosis by morphologic |
| | | evaluation. |
| Liver Biopsy (when | Digital Images | NASH CRN Score: Steatosis grade |
| available) | | (0-3); Inflammation (0-3); Ballooning |
| | | (0-2); Fibrosis stage (0-4) |
| Salivary and Stool | 16s rRNA metagenomic | Taxonomic classification and species |
| samples | sequencing | abundance |
| Plasma, serum | Proteomics and | Metabolite/proteomic profiles |
| | Metabolomics | |
| Genetics | Sanger sequencing from | Whole exome sequencing |
| | buffy coat preparation | |

An omics database 106b may include genome data indicating DNA alterations that cause disease, transcriptome data indicating a subjects RNA regulation profile, metabolome and lipidome data indicating a subjects metabolite levels, metabolic and inflammatory proteome data indicating a subject's serum inflammatory and metabolic protein levels, and spatial metabolomics indicating a subject's metabolite counts per spot data. Non-limiting examples of types of data stored in the omics database 106b (or other subject databases 106) are shown and described in TABLE 2.

TABLE 2
Example Subject Multimodal Omics Data.

| | | | | Molecular and Other | Sample |
|---|---|---|---|---|---|
| Data | Technique | Instrument | Data type | Targets | Form |
| Clinomics | Automated | EPIC and | EHR data, | Patient reported | Electronic |
| | EHR data | Data | clinical | outcome, dietary | |
| | extraction | Warehouse | variables: | and lifestyle info. | |
| | | | NAFLD | | |
| Radiomics | BG-Net and | GE 1.5 T | DICOM | MRI-based hepatic | Electronic |
| | Super U-Net | MRI | files, liver | and body | |
| | | | fat/fibrosis, | composition analysis | |
| | | | fat, muscle | | |
| | | | mass | | |
| Genome | Exome | Illumina | coding | DNA | Blood |
| | sequencing | platform | regions of | | |
| | | | the (~20,000 | | |
| | | | genes). | | |
| | | | (~85%) of | | |
| | | | DNA | | |
| | | | alterations | | |
| | | | that cause | | |
| | | | disease | | |
| Transcriptome | Total High | Illumina | Raw coding | RNA; whole | Serum, Saliva, |
| | Throughput | NExtseq | genes. 10-30 | genome | Urine, Stool, |
| | RNA | | million | transcriptomics | Liver Biopsy |
| | sequencing | | reads/sample | | |
| Metabolome/ | GC/MS, | Agilent 7890 | Metabolites | Glutathione, | Serum, Saliva, |
| Lipidome | LC/MS (RP | Agilent 5977B | levels. Data | Glycocholate, | Urine, Stool |
| | & HILIC) | MS, SCIEX | integration. | etc. >1,000 | |
| | | Triple Quad ™ | | metabolites | |
| | | 7500 | | | |
| | | LC-MS/MS | | | |
| | | QTRAP | | | |
| | | ExionLC ™ | | | |
| Metabolic and | Millipore | Luminex | Serum | Serum proteome and | Serum, Saliva, |
| inflammatory | high | (LabMAP ™) | inflammatory | cytokines associated | Urine, Stool |
| proteome | sensitivity | | and | with metabolism | |
| | multiplex | | metabolic | and systemic | |
| | assay | | protein levels | inflammation (e.g. | |
| | | | | leptin, GLP-1, | |
| | | | | GIP, IL-6) | |
| Spatial | ESI/MALDI | Bruker's | Metabolite | e > 1000 detection | Liver Biopsy, |
| Metabolomics | source with | timsTOF flex | counts per | molecules evaluated. | Intestine |
| | UHR-QTOF | | spot data. | | Biopsy |
| | system | | Spatial | | |
| | | | distribution. | | |

The MRI imaging database 106c may include radiomics data to indicate, for example, a subject's hepatic and body composition, cross-sectional abdominal imaging data to indicate a subjects liver fat fraction, liver stiffness score, presence of portal hypertension, or presence of cirrhosis.

The mobile health database 106d may include patient reported medical data on portable devices, such as mobile phones, tablets, or laptop computers. Non-limiting examples include a subject's vital signs, weight loss, sleep patterns, and physical activity.

In some embodiments, as in the example system 100, the analytics system 101 includes an analytics database 104 that functions as a federated database storing the multimodal subject data from the plurality of subject databases 106. The analytics server 102 may receive and store the subject data obtained from the various subject databases 106 into the analytics database 104. For ease of explanation, the system 100 includes the analytics database 104 from which the analytics server 102 obtains subject data to perform the various processes described herein. However, embodiments need not include such a central or federated database.

Optionally, the system 100 includes one or more conditioned databases 108. The conditioned databases 108 contain conditioned data, which includes preprocessed or “cleaned” instances of the subject data from the subject databases 106. The analytics server 102 or other computing device may execute any number of data conditioning processes, including preprocessing functions that clean, normalize, and format the subject data from the subject databases 106 for downstream operations. Non-limiting examples include data completion functions, data noise reduction functions, data transformation functions, and data normalization functions, among others. The output of such data conditioning processes applied to the subject data may be stored into the one or more conditioned databases 108 and/or into the analytics database 104. The example system 100 includes conditioned databases 108 corresponding to the subject databases 106, though embodiments need not include such a correspondence.

In some embodiments, the analytics server 102 or other computing device may perform data profiling functions in the preprocessing functions. The data profiling functions that generate a subject data profile for the subject. The analytics server 102 may store the data profile into, for example, the analytics database 104, conditioned database 108, subject database 106, non-transitory memory of the user device 114, or other non-transitory storage medium of the system 100.

The analytics database 104 of the example system 100 may function as the federated database storing the multi-model subject data from the subject databases 106 and/or the conditioned databases 108. The analytics server 102 may receive and store the subject data obtained from the various conditioned databases 108 into the analytics database 104. For ease of explanation, the system 100 includes the analytics database 104 from which the analytics server 102 obtains the subject data from the subject database 106 and conditioned database 108 to perform the various processes described herein.

As explained further below, the analytics server 102 may gather the subject data from the subject databases 106 and/or the conditioned databases 108 to store the subject data into the analytics database 104. The analytics database 104 may use the various types of subject data to train the one or more PDTs for the subjects (and any number of optional DTs). In some cases, the analytics server 102 may automatically retrieve or receive the subject data from the subject databases 106 and/or the conditioned databases 108 via the networks 112, at a preconfigured interval or when one or more subject databases 106 or conditioned databases 108 are updated. In some implementations, the user, the subject, or another actor may manually upload or submit, via the networks 112, the subject data from the subject database 106 or conditioned database 108, to the analytics system 101, where a computing device hosting the analytics database 104 may store the received subject data into the analytics database 104. The analytics server 102 may, for example, host an online precision-medicine portal allowing users to upload or otherwise submit the subject data from a subject database 106 or conditioned database 108 to the analytics system 101.

The analytics server 102 of the analytics system 101 may be any computing device comprising one or more processors and software, and capable of performing the various processes and tasks described herein. The analytics server 102 may host or be in communication with the analytics database 104, and receives and processes subject data, experimental results data, experimental configurations, and other types of inputs, which the analytics server 102 may receive from the subject databases 106, conditioned databases 108, and user devices 114, among other potential components of the system 100. Although FIG. 1 shows only a single analytics server 102, the analytics server 102 may include any number of computing devices. In some cases, the computing devices of the analytics server 102 may perform all or portions of the processes and benefits of the analytics server 102. The analytics server 102 may comprise computing devices operating in a distributed or cloud computing configuration and/or in a virtual machine configuration.

The analytics server 102 may execute software programming for a computational software module (sometimes referred to as a “computation module” or “computational module”) that generates, develops, and/or executes predictive models. As an example, the predictive models generated, trained, or otherwise developed by the computational module may include the PDTs for predicting diagnoses, prognoses, treatment strategies, drug responses, and clinical biomarkers. The computational module may generate, develop, and/or execute the PDTs using the subject data in the analytics database 104. As another example, the predictive models generated, trained, or otherwise developed by the computational module may include cohort prediction engines for predicting selected cohorts for clinical trials.

The computational module (or other software programming of the analytics server 102) may generate or develop the PBTs using PBT-related data in the subject data in the analytics database 104.

The computational module may execute various layers of a machine-learning architecture among other functions. The computation module may implement various machine-learning and artificial intelligence algorithms that ingest and integrate the preprocessed data into various predictive models (e.g., PDTs). The computational module may also ingest the PBT-related data, such as experimental results, for developing or updating the predictive models. The computational module may further employ handcrafted models for generating or further developing a predictive model. In some implementations, the computational model employs and fuses both machine-learning models and handcrafted models to generate and develop certain predictive models. Non-limiting examples of the models or techniques may include mechanistic models, stochastic models, and Bayesian networks, among other possible types of machine-learning models or handcrafted models.

The analytics server 102 may execute software programming of layers of a machine-learning architecture providing functions of a biomarker discovery module (sometimes referred to as a “biomarker module”). The analytics server 102 may train the biomarker module to identify potential clinical biomarkers from the subject data of any number of subjects and/or PBT-related data, such as the experimental results data. In this way, the biomarker module may identify biology attributes of the subject in the analytics database 104 for creating the PBTs. Additionally or alternatively, the biomarker module (or other machine-learning model of the analytics server 102) may identify discriminating features for training and applying the various types of machine-learning models described herein.

As mentioned, the analytics server 102 may execute software programming of layers of a machine-learning architecture providing the functions and features of the PDTs. The computation module, for example, may train one or more PDTs for a given subject to generate outputs using certain portions of the subject data of the subject. Each PDT is a predictive model trained to generate outputs predictive of the subject's response to a given stimuli or testing scenario. As an example, the analytics server 102 may train the PDT to predict the likely outcome of administering a proposed drug or other therapeutic to the subject. In training or re-training, the analytics server 102 may apply the layers of the machine-learning architecture defining the PDT on drug-related data and the subject data to output a predicted therapeutic outcome. In some cases, the PDT applies a loss function on labeled data indicating the expected therapeutic outcome and the predicted therapeutic outcome to identify a level of error. The loss function may adjust or tune the hyper-parameters or weights of the PDT's predictive model(s) to reduce the level of error. In some cases, the PDT applies the loss function on experimental results data received from the user device 114, where the experimental results data is based upon the clinician-user performing experimental analysis using the PBT. The analytics server 102 may receive the experimental results data as entered by the clinician into the user device 114 or as generated by a peripheral instrument coupled to the user device 114 or otherwise in communication with the analytics server 102.

Additionally or alternatively, in some implementations, the analytics server 102 may execute software programming of the handcrafted models providing the function and features of the PDTs. Using the user device 114, the clinician may enter configuration instruction defining the handcrafted models executed by the computation module. Over time, the clinician may continually calibrate the parameters or weights of the handcrafted model to reduce a level of error or otherwise improve the predictive accuracy and/or consistency of the PDT's handcrafted model. The clinician may, for example, may continue to calibrate the PDT for a given subject to generate the predictive outputs using all or certain portions of the subject data of the subject. The PDT is a predictive model calibrated to generate the outputs predictive of the subject's response to a given stimuli or testing scenario.

In some implementations, the analytics server 102 may generate and develop the PDT according to handcrafted models and machine-learning models. As an example, the clinician may initialize the programming of the PDT by instructing the PDT to apply the handcrafted model on the subject data for the subject and the therapeutic data (e.g., drug-related data) according to the testing configurations, which indicate the particular testing scenario for a given therapeutic. The handcrafted model may also be applied on the PBT-related data (e.g., experimental results data). The user may continually refine and calibrate the handcrafted model for preconfigured number of runs of the experimental analysis and/or for a preconfigured amount of time. After predetermined condition, the clinician or the analytics server 102 may enable the layers of the machine-learning architecture implementing the machine-learning model for applying the loss function and tuning the parameters, hyper-parameters, or weights of the PDT.

The analytics server 102 may execute softer programming layers of a machine-learning architecture including a machine-learning model functioning as a cohort prediction engine or cohort engine. The layers of the cohort engine may be trained to identify or predict a cohort of one or more subjects based on, for example, the subject data from the subject database(s) 106, the therapeutic testing configurations, and/or the PBT-related data. In this way, the analytics system 102 may improve likely success for precision medicine outcomes, drug discovery and development outcomes, and clinical therapeutic treatment outcomes for individual subjects. In some implementations, for example, the analytics server 102 may apply the PDTs of the subjects against the testing configurations indicated by a user's request for a cohort or a request for testing a proposed therapeutic (e.g., proposed drug). Using the predictive model(s) of the PDTs, the analytics server 102 may predict the cohort of subjects having subject data indicating subjects having, for example, common major genetic attributes, lifestyle attributes, and environmental attributes; where the predictions are made on certain testing configuration, such as safety requirements, drug efficacy requirements, and drug candidate efficacy requirements.

In some implementations, the analytics server 102 may iteratively train layers of a machine-learning architecture for machine-learning architecture of an experiment selector. The analytics server 102 may iteratively train the experiment selector to iteratively select experimental analysis constraints (e.g., PBT experiments) to efficiently construct a comprehensive map of drug efficacy within a cohort of users. Rather than testing all possible combinations of drugs and PDT models, the analytics server 102 (e.g., computational module) may iteratively refine the PBT and PDT models. The analytics server 102 may be trained to optimally prioritize the PBT experiments that would make the greatest contribution to the understanding of drug safety and efficacy. The analytics server 102 and/or the clinician may select or indicate design information (as testing configurations reflecting the experiment design) for conducting the experimental analysis (or experiment) and for creating the PBT corresponding to the PDT.

Following the experiment or at predetermined intervals, the analytics server 102 obtains the experimental results data from using the PBT, and stores as the PBT-related data into the analytics database 104 or other database(s) 106, 108. The computational module may re-train or calibrate one or more predictive models of the analytics system 101. For example, the analytics server 102 and the computational module may obtain the experimental results data, which the computational module may then feed back into to PDT models for retraining the PDT model, which may include applying one or more types of computational models including machine-learning, mechanistic, stochastic, and Bayesian networks, among others. The improved PDTs are then applied for subsequent rounds of the experimental design. This active learning approach may beneficially generate improved PDT models and PBT biomedical models. For instance, a loss layer of the PDT's machine-learning architecture (or other type(s) of computational model(s) executed by the analytics server 102) includes a loss function. The loss function determines a level of error of the computational model by determining a distance between the predicted output (e.g., predicted experimental outcome, predicted cohort), against the corresponding observed outcome generated by the PDT model (e.g., machine-learning, mechanistic, stochastic, and Bayesian networks), as indicated by the experimental results data. The loss function may tune the hyper-parameters or weights of the PDT model based upon the level of error, where the analytics server 102 may retrain the particular PDT model by applying the PDT model on the subject data and the level error, among other potential inputs.

### Example PBT Experimental Platform

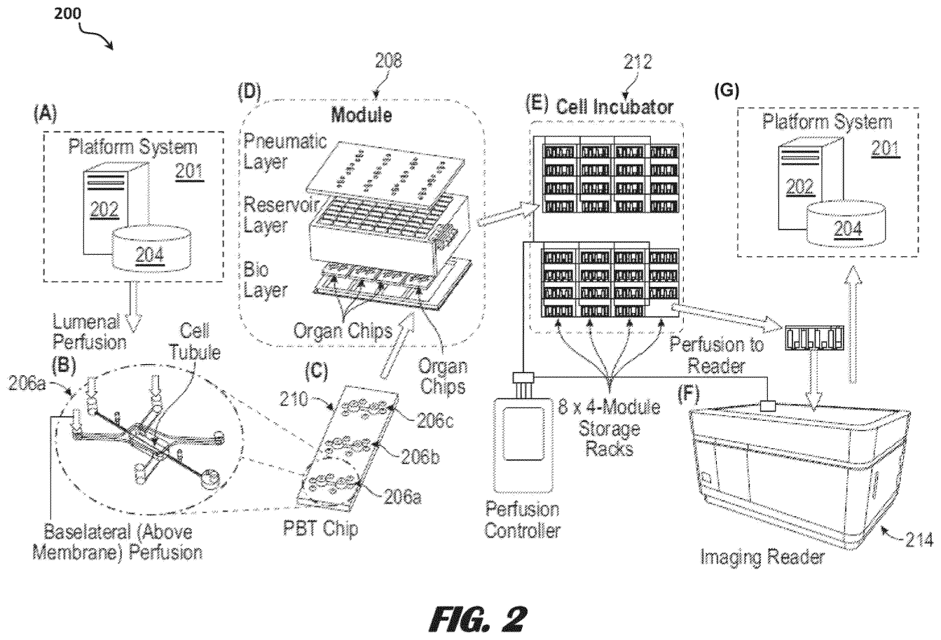

FIG. 2 shows data flow among devices of a system 200 for PBT development and experimentation using predictive PDTs, according to an embodiment. The system 200 includes an analytics system 201 having analytics servers 202 and analytics databases 204 for developing the PDTs among other predictive models. The system 200 further includes perfusion modules 208 housing any number of microfluidic chips 210 (sometimes referred to as “PBT chips” or “organ chips”). A microfluidic chip 210 includes any number of chambers 206a-206c (generally referred to as chambers 206 or a chamber 206). The system 200 further includes a cell incubator 212 that receives the perfusion modules 208 and an inline imaging reader 214 coupled to the cell incubator 212 via a perfusion controller. Moreover, FIG. 2 depicts the data flow for configuring an experimental analysis (or experiment) using, for example, predictive models (e.g., PDTs, cohort predictor, experiment selector) and other types of data; performing the experiment using PBTs created according to the PBTs and experimental configurations; and capturing experimental results data from running the experiment. The analytics system 201 is employed on the frontend of the data flow to design the experiment, such as developing and executing predictive models for selecting PBTs and other relevant information, such as compound and subject data.

(A) The analytics server 202 retrieves and executes the predictive models from the analytics database 204, including the PDTs for the subjects and the subjects' subject data. In some implementations, the analytics server 202 executes a cohort predictive model for predicting and selecting the cohort of subjects for the experiment. In some implementations, the analytics server 202 executes experiment selector model to identify a type of experiment to perform given the clinician's testing configurations and the subject data for the subjects. Optionally, the experiment selector may be trained to identify and output predicted or suggested compounds or other types of therapeutic parameters. In some cases, this information may be included in the testing configurations used by the analytics server 202 to apply the predictive models of the PDTs to predict the outcomes.

The analytics server 202 transmits, or otherwise outputs, predicted outcomes and related information to a user interface operated by a clinician. The outputs of the predictive models aid the clinician in designing and setting up the experimental analysis. The analytics server 202 may execute the predictive model of the trained PDT for the subject by applying the PDT on the subject data according to the various testing configurations. The testing configurations may include PBT-related data that indicate real-world PBT configurations and/or real world compound configurations, which a clinician will reference to setup a real world instance of the experiment (corresponding to the digital instance of the experiment modeled by the PDT). After executing the PDT to generate the predicted outcome, the analytics server 202 may provide the output of the predicted outcome and the PBT configuration information to the user interface, which may be presented at a user device of the clinician.

(B)-(C) The clinician uses the outputs of the analytics system 201, including information related to the PBT design, for setting up and performing the experiment. The clinician may take samples from the cohort of subjects and prepare the PBT according to the information received from the analytics system 201. As an example, the clinician may take samples from the subjects for creating the PBTs as physical cells or organoid of the subjects on one or more microfluidic chips 210. The microfluidic chip 210 includes one or more culture chambers 206, and each chamber 206 comprises a perfusion channel and an injection port. When setting up the PBT, the subject's cells are introduced through the injection ports or added prior to assembly of the microfluidic chip. Bubble traps are positioned on fluid paths to prevent air from entering the culture chambers 206. The chambers 206 provide luminal perfusion, basolateral perfusion, and cell tubules.

(D) The clinician introduces the microfluidic chips to the microfluidic chip 210 perfusion modules 208. The perfusion module 208 houses the one or more microfluidic chips 210. In addition, the perfusion module 208 includes fluid reservoirs and contains a bio layer, a reservoir layer, and a Pneumatic or hydraulic pressure layer.

(E) The perfusion modules 208 are stored in a CO₂ cell incubator 212. The incubator 212 contains storage racks (e.g., 8×4 storage racks) for the perfusion modules 208, as well as a pneumatic or hydraulic pressure gas pump located outside the incubator 212. A perfusion controller coupled to the cell incubator 212 manages the functions of the cell incubator 212.

As an example, a single microplate-footprint module 208 may be divided into 4 sections of 3 experiments (e.g., 4 microfluidic chips 210, each with 3 chambers 206 for cells), thereby supporting high-throughput of 12 experiments per well plate. Further, the integrated perfusion controller allows fluid passage to be controlled in each of the 4 microfluidic chips 210. The pneumatic or hydraulic pressure control system of the cell incubator 212 may include high-resolution digital pressure controllers, along with on-chip flow restrictors and passive mixing channel, which produces user-programmable drug concentrations, for up to, e.g., 768 PBTs in the single incubator 212. The microfluidic chip 210 is configured to minimize drug absorption losses. The microfluidic chip 210 may be constructed from injected molded hard plastics and, in some embodiments, is not constructed with any silicone. In addition, in some embodiments, the microfluidic chip 210 and/or the cell incubator 212 includes interfaces for relatively easily coupling to confocal high content imaging systems 214.

(F) A laboratory instrument may capture or generate the experimental results data based upon the outputs of the cell incubator 212. For example, an inline imagining system imaging reader 214 is associated with the incubator 212 to generate results data containing, for example, live cell readouts. Additionally or alternatively, the clinician may input the experimental results data to the user device.

In some cases, the PBT-testing platform components may beneficially assess each PBT rapidly in multi-dose, multi-drug treatment to evaluate a proposed therapeutic efficacy and safety. The PBT data (e.g., PBT preparation data, results data) will include, for example, secretome, live cell, metabolomics or RNA-Seq, and/or endpoint IF imagery. This PBT-related data may be uploaded to the analytics system 201. In some cases, the PBT-related data may be combined with clinical measurements and omics data for upload to the analytics system 201.

(G) The user device or laboratory instrument may transmit, upload, or otherwise provide the experimental results data to the analytics system 201. The analytics server 202 may receive and store the results data into the analytics database 204. The predictive models used to design and prepare the experiment include loss functions that, when executed by the analytics server 202, determine a level of error between the prediction output generated by the particular predictive model and certain observed values in the results data. In some cases, after the analytics server 202 receives the results data, the analytics server 202 may further apply the one or more loss functions of the relevant predictive models to determine the corresponding level of error. For certain predictive models, the loss function may include programming for re-training or tuning the predictive model by adjusting or tuning the hyper-parameters or weights of the predictive model based upon, for example, the level of error, experimental results data, and subject data, among other potential types of data. In training, the analytics server 202 may determine that that any of the predictive models described herein are satisfactorily trained or output a sufficient result if the level of error satisfies a training threshold, a similarity threshold, or other threshold value.

As mentioned, the PBT data (e.g., PBT preparation data, results data) will include, for example, secretome, live cell, metabolomics or RNA-Seq, and/or endpoint IF imagery. This PBT-related data may be uploaded to the analytics system 201. In some cases, the PBT-related data may be combined with the clinical measurements and the omics data for upload to the analytics system 201. The analytics server 202 may incorporate the PBT-related data into the predictive models that the analytics server 202 executed when designing the experiment performed in the system 200.

The number of experiments per PBT may correspondingly improve accuracy of the various types of data used for the predictive models of the PDTs. This beneficially shifts the currently practiced whole population approach of disease treatment towards an approach of algorithmically refined and validated therapeutic treatments, customized for optimized efficacy and/or drug safety to specific subject sub-populations. Likewise, the potential benefit on human health and the economic aspect would be a significant improvement in cohort selection and cost reduction of clinical trials.

### Example Processes

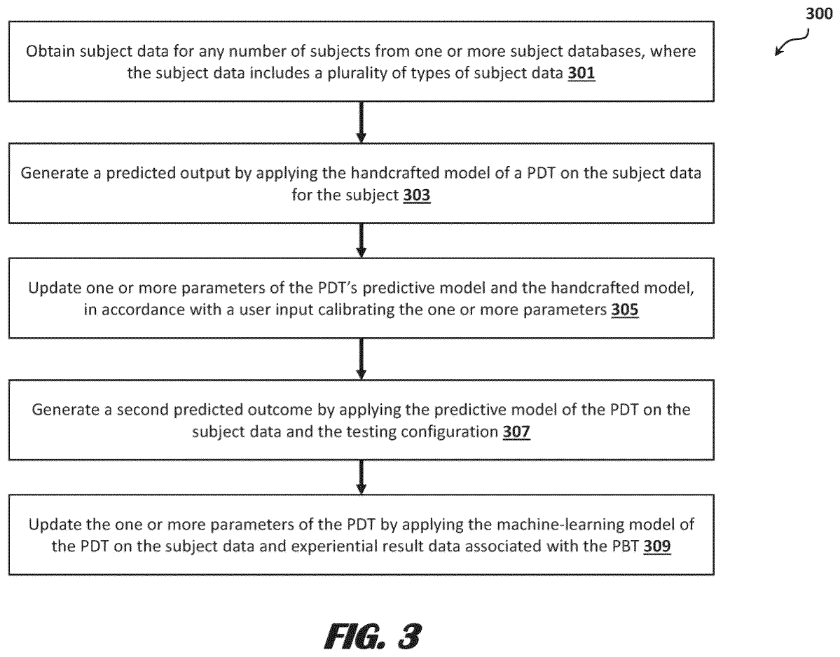

FIG. 3 shows operations of a method 300 for developing PDTs for predicting and using PBTs in experimental analysis, according to an embodiment. A computer (e.g., analytics server 102) may develop predictive models of PDTs, and correspondingly develop the biomodel PBTs using the PDTs. The computer trains one or more PDTs for a given subject, using subject data of a given subject stored in a database (e.g., analytics database 104). The predictive models of the PDTs are trained, according to handcrafted models and machine-learning models, for predicting experimental outcomes when executed according to testing configurations of an experimental analysis, such as measuring disease progression or predicting therapeutic efficacy, were the experimental analysis applied to the subject in the real world. The trained PDTs are applied on experimental testing configurations, representing, for example, disease attributes or proposed therapeutic (e.g., drug, treatment), to generate the predicted experimental outcome. The method 300 includes both handcrafted models and machine-learning models for the PDTs, though embodiments may include only handcrafted models or only machine-learning models.

In operation 301, the computer obtains subject data for any number of subjects from one or more subject databases, where the subject data includes a plurality of types of subject data. The databases may include one or more types of databases, such as the analytics databases 104, subject databases 106, and/or conditioned databases 108. The computer may obtain the subject data by retrieving the subject data at a given interval or in response to a user command to retrieve the subject data. Alternatively, the computer may obtain the subject data by receiving pushes, replications, or other form of update from the database.

In some cases, the computer obtains the subject data of only a single subject. Alternatively, the computer may obtain the subject data of a plurality of subjects, such as a population of subjects or a subset cohort of subjects from the broader population.

In operation 303, the computer generates a predicted output by applying the handcrafted model of a PDT on the subject data for the subject. The predictive model of the PDT includes programming configured to generate the predicted outcome of a testing configuration according to the handcrafted model.

In operation 305, the computer updates one or more parameters of the PDT's predictive model and the handcrafted model, in accordance with a user input calibrating the one or more parameters. As an example, a clinician may run multiple iterations of an experiment. Prior to each iteration, the clinician executes the handcrafted model programming of the predictive model, to generate a predicted experimental outcome (or other type of predicted value).

In some implementations, the clinician may employ both handcrafted models and machine-learning models for training and tuning the predictive model. The handcrafted model may be used as an initial basis for the predictive model, and the clinician may adjust the parameters to calibrate the predictive model. After a number of iterations or other triggering conditions (e.g., amount of subject data, amount of experimental result data), the computer may apply a machine-learning model to the same or new predictive model, which includes pattern recognition functions (e.g., clustering, outlier detection, stochastic modeling, Bayesian networks) and a loss function for identifying features (or attributes of the subject) and adjusting parameters or hyper-parameters of the predictive model.

In some embodiments, the handcrafted modeling approach may be applied solely during the initial development (e.g., adjusting the weights or parameters) of the PDT's predictive model. The handcrafted model may be applied against a plurality of subjects to generate a broader, generalized predictive model for a broader population of subjects. In such embodiments, the computer may store this predictive model as a distinct, generalized predictive model and generalized PDT for the collection of subjects.

Correspondingly, in some embodiments, the computer may apply the machine-learning model on the subject data of only one subject or a small cohort of subjects to generate a customized predictive model for a tailored PDT. In such embodiments, the computer may store this predictive model as a distinct, customized predictive model for a customized PDT.

In operation 307, the computer generates a second predicted outcome by applying the predictive model of the PDT on the subject data and the testing configuration. The computer may switch to applying the machine-learning approach to tuning and/or generating the predicted outcomes. In operation 309, the computer updates the one or more parameters of the PDT, by applying the machine-learning model of the PDT on the subject data and experimental result data for the subject associated with a PBT. The loss function, for example, measures a difference between the predicted experimental outcome for a given iteration and the actual experimental result data produced from testing the PBT corresponding to the PDT. The loss function may then adjust this level of error and apply the predictive model on the next iteration of the experiment.

### Example Implementations

With reference again to FIG. 1, the components of the system 100 may be employed in various example implementations mentioned below for various benefits.

In some embodiments, the analytics server 102 may train and tune predictive models and design experimental analyses (e.g., experiments) using PBTs. The recent wide adoption of high-throughput and high-content screening has dramatically increased the availability of information on chemical compounds affecting specific molecular targets under different conditions. However, it is infeasible for clinicians to perform experiments for all possible combinations of drugs, targets, and conditions. To address this problem, embodiments may leverage the PDT modeling to guide efficient PBT experimentation. Embodiments include a server (or other computing device) configured for executing programming for computational modeling techniques for various predictive models, which may include applying (or reapplying in training) one or more types of computational models, such as machine-learning, mechanistic, stochastic, and Bayesian networks, among others. In some implementations, the server may develop active learning predictive models of a cohort selection engine (“cohort selector”) for optimized selection of subjects that receive a specific drug, which may ingest and employ the models of the PDTs. In some implementations, the server may develop predictive models of an experiment selection engine (“experiment selector”) for iteratively predicting and selecting the PBT experiments with the comparatively highest predicted information content from a massive number of potential experiments. In some implementations, the server may develop predictive models and information capture routines of compound selection that predicts or suggests drug compounds and effectively generates compound libraries and small molecule screenings, which beneficially supports higher throughput PBT experimental studies and reduces the need for trial-and-error for developing therapeutics or for immediate treatment needs.

In some embodiments, developing the analytics database 104 and subject profiles creates a deeply phenotyped and genotyped cohort of NAFLD patients and a collection of multimodal datasets and cells. The analytics server 102 may ingest subject data, including deep clinical and multi-modal datasets (e.g., TABLE 1 and/or TABLE 2), for subjects enrolled in various clinics or treatment centers having subject databases 106 containing the subject data. The clinician may focus on diverse subjects with less than 35 BMI, exhibiting NAFLD (>5% fat) with or without Type 2 Diabetes, which the analytics server 102 may store into the conditioned database 108 as subject profiles of a subject cohort. The clinician develops two sub-cohorts, subjects with advanced fibrosis compared to subjects with no or minimal fibrosis. These datasets of the subject databases 106 or conditioned databases 108 include, for example, the clinomics, radiomics, subject-reported measures, and multimodal omics data. The clinician may employ the PDTs in parallel projects to develop a precision medicine platform for NAFLD.

In some embodiments, the analytics server 102 develops and implements preprocessed, multimodal data warehouse as the analytics database 104 from the subject databases 106 and conditioned databases 108. The analytics server 102 may reference the analytics database 104 to support the creation of PDTs.

To develop the analytics database 104, the analytics system 101 may collect subject data from subjects (e.g., sub-cohorts of NAFLD subjects) using the research data warehouses of the subject databases 106 or conditioned databases 108, which include subject datasets such as those of TABLE 1 and/or TABLE 2. The conditioned databases 108 integrate and harmonize EHRs from various data sources (e.g., disparate subject databases 106).

The analytics server 102 may include and execute an API connection for user devices 114 to access the PBT-related data maintained by the analytics system 101. The analytics system 101 may develop the analytics database 104 as a federated data warehouse, accessible to the user devices 114 or other data sources via web portal. The analytics server 102 may execute webserver software hosting a cloud computing web-app portal for the user devices 114 or other data sources to interact with the analytics database 104, which may host, for example, multimodal omics data derived from subjects and PBTs that are not maintained by the analytics system 101. In this way, the analytics system 101 establishes a networked or computing based link between the analytics system 101, data sources (e.g., subject databases 106, conditioned databases 108), subjects, and other actors (e.g., honest broker services). The various entities and computing devices may interact with the analytics system 101 using the API as a centralized interface.

In some embodiments, the analytics server 102 may develop and host (via the API) a clinical natural language processing (NLP) pipeline for clinical phenotyping using the subject databases 106, which may extract the clinical information relevant to NAFLD patients from unstructured clinical notes (or other unstructured data) of the subject databases 106. In training and developing the NLP, the clinician and analytics server 102 maps the extracted information to biomedical ontologies (controlled vocabularies), such as, UMLS and LOINC for consistent representation of clinical information. The analytics server 102 may apply the NLP for ingesting the subject data from the subject database 106 and for developing the conditioned database 108 and/or developing the analytics database 104.

The analytics server 102 may collect and process data derived from NAFLD subjects, such as quantifying genomic and transcriptomic data. The clinician or analytics server 102 may deep-mine omics data to extract/construct discriminating features for the predictive models, where the features reflect, for example, the signaling states of cells for following computational discovery and PDT modeling. The analytics system 101 may develop and deploy pipelines, and provide application programming interfaces (APIs) to output various types of data for downstream computational discovery or modeling, such as the subject data for training the predictive models of the PDTs.

The analytics server 102 may train the predictive models of the PDTs using knowledge-based handcrafted models, data-driven machine-learning models, or both. For the data-driven predictive models approach, the analytics server 102 may apply the model of the PDT on subject data from the analytics database 104 (or other data source) and PBT data, where the various types of data are collected from a cohort of subjects and from publicly available clinical trial data. The analytics server 102 outputs the trained PDT based on historic knowledge but customized to specific subject data.

In some embodiments, the analytics server 102 may begin developing the PDT by implementing a handcrafted model (historic knowledge-based model) of disease progression within the analytics system 101. The analytics system 101 may develop the handcrafted model of the PDT to establish an initial mechanistic model to predict clinician-configured disease progression stages, with a limited set of experimentally measurable outputs, linked by a set of initially clinician-configured knowledge-derived kinetic rate constants fit from literature-derived longitudinal data (e.g., subject data from multiple subjects). The analytics server 102 may apply a Bayesian optimization function, allowing the clinician to refit parameters of the handcrafted model and identify mappings of model-states to additional multimodal outputs (e.g., genomics, metabolomics) using the primary subject's data from the analytics database 104. The result will be, for example, a PDT having a handcrafted model developed as an algorithmic-system of differential equations describing disease progression of a generic subject.

The analytics server 102 may then execute layers of the computational model architecture to develop the predictive model of the PDT for patient-specific customization through computational modeling techniques drawing on strategies for causal inference, such as machine-learning, mechanistic, stochastic, and Bayesian networks, among others. In some embodiments, for example, the analytics server 102 may apply the predictive model configured for aggressive feature selection, prescreening for significantly correlated individual features, and lasso regularization on the remaining feature set. In addition to configuring feature selection of the PDTs, the analytics server 102 may execute deep learning methods to construct/identify discriminating features of the PDTs optimal for predicting patients' drug responses and disease progression.

In some implementations, the analytics server 102 may tune, retrain, and validate the predictive models of the PDTs by comparing predictive outputs of the baseline, non-personalized, knowledge-based handcrafted model against the outputs of other handcrafted models. The analytics server 102 may evaluate a level of error by cross-validating accuracy of the handcrafted model of the PDT in stratifying subjects from the primary subject cohort. Using the comparative predictive outputs, the analytics server 102 may identify differences in predicted disease progression outcomes. Subsequently or contemporaneously, the analytics server 102 may similarly validate the predictive outputs of the patient-specific machine-learning models of the PDTs against the experimental results generated from using the PBTs for patient-specific disease progression outcomes conditional on treatment.

As mentioned, the subject experimental representation is a biomedical model (also referred to as a “subject PBT” or “MPS”) can be used to understand normal and abnormal functions and provide a basis for preventive or therapeutic intervention in human disease. In some embodiments, the biomedical model is a patient biomimetic twin (PBT). A PBT can also be referred to as a microphysiology system (MPS) or an organ on chip (OoC). For a given subject, both a PBT and a PDT may be generated using data from the same subject, such that the PBT may be created as the biomedical (or otherwise physical) model of the subject and the PDT may be generated to computationally model the same subject. In some embodiments, the subject experimental representation may include 3D-layered cells. The 3D layered cells are produced by a combination of sequential cell layering and cell-to-cell self-organization of specific cell types. The cell types migrate and self-assemble into distinct layers forming tissue structures. In some embodiments, one or more cell types are introduced through the injection port. In some embodiments, the one or more cell types are organoids, induced pluripotent stem cells (iPSC)-derived cells or primary cells.

For example, the 3D layer of cells includes a functional human liver from iPSC-derived cells. To produce iPSC-derived cells, blood samples are collected from subjects. The iPSC cells are produced from monocytes to generate iPSC-derived cells. The iPSC cells are programmed into liver specific cells, which may be iPS-Hepatocytes, iPS-Cholangiocytes, iPS-Endothelial cells, iPS-Stellate cells, and/or iPS-Macrophages (also referred to as iPS-Kupffer cells). It should be appreciated that the PBT may comprise or involve one or more connected organ MPS models (e.g., liver MPS, pancreatic islets MPS, etc.) and/or may include one or more types of biomedical models that may function as the PBT(s) described herein.

In some embodiments, the 3D layer of cells is housed in a microfluidic chip (e.g., microfluidic chip 210). The microfluidic chip comprises at least one chamber comprising perfusion channels and injection ports. In some embodiments, the microfluidic chip comprises one or more perfusion channels and one or more injection ports. In some embodiments, the microfluidic chip has medium flowing through the microfluidic chip. In some embodiments, one or more microfluidic chips are located in a perfusion module comprising a bio layer, a reservoir layer, and a pneumatic or hydraulic pressure layer. In some embodiments, one or more of the perfusion modules are placed in a CO₂ cell incubator. In some embodiments, the cell incubator comprises an in-line imaging system.

In some embodiments, the PBT's are designed to recapitulate key structural, functional, and clinical characteristics of a disease allowing subject-specific studies of mechanisms of disease progression and drug action. The PBT can be used for any experimental analysis. The raw experimental data is produced from the PBTs using a user device 114 or other instrument for generating machine-readable data for the analytics server 102. Non-limiting examples include DNA, RNA, protein, and cell functional analysis. In some cases, the PBT can be used to analyze toxicity and proliferation. The experimental data generated and provided to the analytics server 102 from the PBTs may include secretome data, live cell data, metabolomics data, RNA-seq data, or endpoint immunofluorescence imaging data.

The PDT and PBT are used in combination to predict and/or validate, for example, drug efficacy, ADME-Tox, clinical trials, and therapeutic strategies, among other beneficial uses. In some embodiments, the PDT includes the predictive models as a digital representation of how the biology of a particular subject or subject cohort would respond. The analytics server 102 may dynamically integrate the multimodal clinical data acquired over time from the subject databases 106 and/or conditioned databases 108, and individual subjects to generate or update the PDTs. Example datasets for the subject data are shown in TABLE 1 and TABLE 2. The analytics server 102 may generate or update the PDTs using machine-learning models (e.g., statistical, data-driven models) and/or handcrafted models (e.g., mechanistic, knowledge-based models). The PDTs may predict outcomes of, for example, disease progression in the subject biology and/or the impact of specific therapeutics on the subject biology.

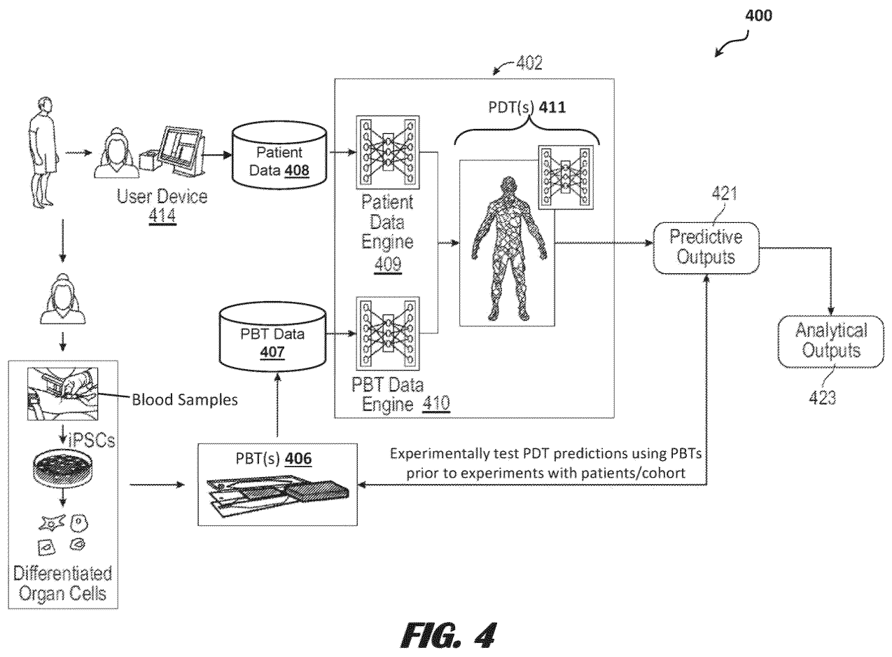

FIG. 4 shows operations and data flow amongst components of a system 400 for generating and managing patient data using machine-learning architectures, according to an example embodiment. The system 400 includes analytics servers 402 (e.g., analytics servers 102, 202), user devices 414, PBT data databases 407 (e.g., analytics databases 104, 204), and patient data databases 408 (e.g., analytics databases 104, 204; subject data databases 106; conditioned subject data databases 108). The analytics server 402 executes software programs or routines that define or perform the operations of one or more functional engines, such as a patient data engine 409, a PBT data engine 410, and PDTs 411 having one or more predictive models. For ease of description and understanding, the system 400 is depicted and described with one instance of certain components, such as the analytics server 402, patient data database 408, and PBT data database 407, though embodiments may include any number of components. The PBTs 406 are created from cells or other physical samples from specific corresponding patients, and the PDTs 411 are generated using the patient-related data from the same patients. As depicted in FIG. 4, the PBTs 406 may be used to test predictions generated from predictive models of the PDTs 411 and then the results can be updated in or used to update the PDTs 411; and the PBTs 406 may be used to test the predicted treatments output from the PDT 411 and/or PBT 406 models in order to, for example, test a drug in the PBT 406 before treating a patient or cohort.

Embodiments may comprise additional or alternative components or omit certain components from those of FIG. 4 and still fall within the scope of this disclosure. It may be common, for example, to include multiple analytics servers 402. Embodiments may include or otherwise implement any number of devices capable of performing the various features and tasks described herein. For instance, FIG. 4 shows the analytics server 402 as a distinct computing device from the patient data database 408. In some embodiments, the analytics server 402 includes an integrated patient data database 408.

The patient data database 408 contains various types of patient data (sometimes referred to as “subject data” or “conditioned data”). The patient data database 408 may be hosted on one or more computing devices comprising hardware components (e.g., non-transitory machine-readable storage media, processor) and software components (e.g., database management system (DBMS)) capable of performing the various tasks and processes described herein. Similarly, a computing device may host patient data databases 408 or other types of databases, such as the PBT data database 407. In some implementations, the patient data database 408 includes conditioned data or a conditioned database (e.g., conditioned database 108) containing conditioned data, where the conditioned data includes preprocessed or “cleaned” instances of the patient data. The patient data database 408 receives, stores, manages, and queries various types of patient data. Non-limited examples the patient data includes clinomics, genomics, metabolomics, proteomics, transcriptomics, epigenomics, and microbiomics, among other types of patient data. The user device user device 414 or analytics server 402 (or other device of the system 400) references the patient data in the patient data database 408 and generates or updates the PDTs 411.

The PBT data database 407 contains various types of PBT data, such as data indicating the properties or information describing the PBTs 406 and experimental results data indicating various types of information related to PBT-based experiments and results of conducting the PBT-based experiments. The PBT data database 407 may be hosted on one or more computing devices comprising hardware components (e.g., non-transitory machine-readable storage media, processor) and software components (e.g., database management system (DBMS)) capable of performing the various tasks and processes described herein. Similarly, a computing device may host PBT data databases 407 or other types of databases, such as the patient data database 408. The PBT data database 407 may be a distinct database from the patient data database 408, or the PBT data database 407 may be integrated into the same database as the patient data database 408. The patient data database 408 receives, stores, manages, and queries various types of PBT data. Non-limited examples the PBT data includes clinically relevant data, genomics, metabolomics, proteomics, transcriptomics, epigenomics, microbiomics, and experimental results data, among other types of data. The user device user device 414 or analytics server 402 (or other device of the system 400) references the PBT data in the PBT data database 407 to design or update the PBTs 406 or to generate or update the PDTs 411.

The user device 414 (e.g., user device 114) includes a computing device that allows an administrator-user (e.g., clinician, medical provider, researcher) to interact with therapeutic modeling services of the system 400. Administrators of the system 400 include any users responsible for capturing various types of information samples from patients and/or managing the various machine-executed processes and tasks described herein. Non-limiting examples of administrators in the example embodiment include clinicians, researchers, and healthcare providers (e.g., doctors, nurses), among others.

The user device 414 may include any computing device comprising hardware (e.g., non-transitory machine-readable storage media, processors) and machine hardware-executed software components capable of performing the processes and tasks described herein. Non-limiting examples of the user device 414 may include a personal computer (PC) (workstation computer, laptop), tablet, and smartphone, among other types of electronic devices capable of performing the functions of the user device 414 described herein. The user device 414 comprises, or couples to, peripheral devices for receiving the user inputs, such as user I/O devices (e.g., keyboard, mouse, monitor), allowing the administrator to interact with the user device 414 and the various features of the system 400. In some cases, the peripheral devices may include electronic measurements devices or instruments that obtain (e.g., receive, generate) patient data (e.g., biological measurements of a patient) or PBT data (e.g., properties of PBTs 406; experimental results data when conducting PBT-based experiments on certain PBTs 406). Non-limiting examples of such electronic instruments include an image reader, microscope, live cell image reader, sequencing system, thermal cycler, and gel and blot readers, among others. The administrator may enter patient-related information as patient data into a user interface of the user device 414 and/or enter PBT-related information as PBT data into the user interface of the user device 414. The user device 414 transmits or otherwise stores the patient data into the patient data database 408 and the PBT data into the PBT data database 407.

In the example embodiment, one or more administrators of the system 400 capture various types of patient information and/or patient samples, used to generate, update, or employ PDTs 411 for digitally modeling patients and PBTs 406 for physically modeling patients. The patients may enroll with the system 400 (e.g., enroll with healthcare clinic; enroll or permit analytics service). For example, an administrator of the system 400 operates the user device 414 to capture patient-related information for a new patient, where the administrator enters the patient information as patient data (sometimes referred to as “subject data”) into the user interface of the user device 414. Additionally or alternatively, an electronic measurement device (e.g., thermometer, electronic heat rate cuff) coupled to the user device 414 or the patient data database 408 generates certain types of patient data. The user device 414 or the electronic measurement device transmits or otherwise stores the patient data into the patient data database 408.

An administrator captures or creates patient samples (e.g., blood samples, saliva swabs, skin samples) and/or intermediate physical items (e.g., induced pluripotent stem cells (iPSCs), differentiated organ cells, organoids), which the administrator uses to create one or more PBTs 406 corresponding to the patients. Using these patient samples (or other physical items), the administrator prepares one or more PBTs 406 that physically model the biology of the patient and are expected to respond to experimental stimuli introduced by the administrator to the PBT 406 during PBT-based experiments according to experiment parameters. As an example, an administrator draws the patient's blood to collect patient blood cells, which the administrator then uses to create iPSCs, from which the administrator generates differentiated organ cells and/or creates organoids from patient tissue samples or iPSCs. Using these samples or the created physical items, the administrator may then generate the PBTs 406 in non-animal models (NAMS), such as microphysiology systems (MPS).

The administrator or an electronic measurement device of the system 400 may identify, measure, determine, or otherwise capture various types of information related to the PBTs 406, such as the properties of each PBT 406 and any experimental results generated using certain PBTs 406. The administrator may enter the PBT-related information into the user interface of the user device 414 as the PBT data, and/or an electronic measurement device may output experimental results data for the PBT data. The user device 414 or the electronic measurement device then stores the PBT data for one or more PBTs 406 into the PBT data database 407.

The analytics server 402 of the analytics system 101 may be any computing device comprising one or more processors and software, and capable of performing the various processes and tasks described herein. The analytics server 402 executes the software programming of the functional engines (e.g., patient data engine 409, PBT data engine 410, predictive model of the PDT 411) using the PBT data, PDT data, or other types of data. The software routines of the functional engines may include operations of one or more computational models. The executable code defining the functional engines instructs the analytics server 402 to perform the computational models, which may include handcrafted models or machine-learning models (e.g., predictive machine-learning models) as layers of a machine-learning architecture. The analytics server 402 may contain, generate, train, or develop the various types of computing computational models by taking, as inputs, various multi-modal datasets stored in one or more databases of the system 400.

The analytics server 402 generates or updates the data or functions of the PDT 411 associated with a patient by executing the patient data engine 409, where the analytics server 402 uses the outputs of the patient data engine 409 for updating the PDT 411. In some cases, the analytics server 402 updates the PDT 411 by executing the patient data engine 409 and the PBT data engine 410, where the analytics server 402 algorithmically combines or integrates the outputs of the patient data engine 409 and the PBT data engine 410.

The patient data engine 409 of the analytics server 402 performs various functions using the patient data database 408. In some implementations, the PBT data engine 410 retrieves certain types of PBT data for a set of one or more patients from the PBT data database 407 and performs certain operations for processing or converting the retrieved PBT data to a format compatible with the outputs of the patient data engine 409 or otherwise compatible with the types of data of the PDT 411.

In some implementations, the PBT data engine 410 may train, tune, or develop one or more machine-learning models for the PBT data. As an example, the PBT data engine 410 may ingest the PBT-related data, such as experimental results data, to develop or update PBT data-selection models (e.g., biomarker discovery models) that identify discriminating features in the PBT data and/or the PDT data, which may be received as feedback from predictive outputs 421 or the experimental results data from the PBT data database 407. As another example, the PBT data engine 410 may ingest the PBT-related data to develop or update the predictive models of the PDTs 411. The PBT data engine 410 trains, tunes, or develops the one or more machine-learning models by executing certain PBT-related models taking the various types of data, such as PBT data (e.g., experimental results data), PDT data, and predictive outputs 421, among other types of data for training PBT-related machine-learning models. The analytics server 402 algorithmically combines or integrates the outputs of the patient data engine 409 and the PBT data engine 410 to generate or update the PDT 411.

The analytics server 402 executes the predictive models of the PDTs 411 to generate the predictive outputs 421 containing predicted experimental result data. The administrator may, for example, enter various configuration inputs to the user device 414 to configure a digital experimental having certain experiment parameters. The predictive model of the PDT 411 then performs a digital instance of the experiment, using the PDT data and experiment parameters defining the experiment. The PDT 411 then generates the predictive outputs 421 indicating the predicted experimental result, in the form of the experimental result data.

The administrator may then test the predictive outputs 421 from the PDTs 411 by conducting a PBT-based experiment using the PBTs 406 corresponding to the PDTs 411 involved in the digital instance of the experiment. The administrator creates and configures the PBTs 406 according to the experiment configurations and parameters. The administrator conducts the PBT-based experiment on one or more PBTs 406 and stores the experiment results as PBT-related information in the PBT data database 407. In some cases, the administrator enters certain experimental results into the user device 414 in the form of experimental results data for the experiment conducted on the one or more PBTs 406. The user device 414 transmits or otherwise stores the experimental results data into the PBT data database 407 for the one or more PBTs 406. Additionally or alternatively, an electronic measurement device monitors, generates, or otherwise captures experimental results data for the experiment conducted on the one or more PBTs 406. The electronic measurement device may transmit or store the experimental results data to the user device 414 or the PBT data database 407 for the one or more PBTs 406.

The administrator may instruct the analytics server 402 to execute the patient data engine 409 and/or the PBT data engine 410 to update the predictive models of the PDTs 411 (or other machine-learning models). In some cases, the analytics server 402 updates the PDTs 411 based on differences between the observed experimental results data generated from the PBT-based experiments and the predictive outputs 421 containing the predicted experimental results produced by the PDTs 411 for the digital instance of the experiment. In this way, the administrator may adjust or finalize experiment parameters and/or hypothesized predictions for a proposed, real-world, clinical experiment, before the administrator conducts and applies the real-world experiment using an actual patient or patient cohort.

The administrator, analytics server 402, or user device 414 may generate or determine certain analytical outputs 423 that may be referenced, outputted, or stored as actionable knowledge. The analytical output 423 may be generated or determined based upon, for example, the experimental results data generated from the PBT-based experiment, the predictive outputs 421 generated from the PDT-based digital experiment, or real-world experiment data generated from conducting the real-world experiment on actual patients. The analytics outputs 423 may include, for example, actionable knowledge, such as toxicology, disease progression, drug testing, and clinical trials, among other forms of information generated or extrapolated from the experimental results data generated from the PBT-based experiment, the predictive outputs 421 generated from the PDT-based digital experiment, or real-world experiment data generated from conducting the real-world experiment on actual patients.

Embodiments include a system having one or more computers with at least one processor and non-transitory machine-readable medium storing processor-executed instructions to perform certain operations or a computer-implemented method.

In embodiments, a computer comprises at least processor and is configured to obtain subject data for a subject from one or more subject databases. The subject data includes a plurality of types of subject data. The computer may generate a predicted output by applying a model of a patient digital twin on subject data for the subject. The patient digital twin is configured to generate a first predicted outcome of a testing configuration. The computer may update a parameter of the model according to a user input. The computer may generate a second predicted outcome by applying the patient digital twin on the subject data and the testing configuration. The patient digital twin for the subject contains biomimetic twin configuration data associated with a patient biomimetic twin for the subject. The patient biomimetic twin includes an experimental representation of the subject according to the biomimetic twin configuration data. The computer may obtain experimental result data for the subject associated with the patient biomimetic twin for the subject and the testing configuration. The computer may update the parameter of the patient digital twin to train the patient digital twin, by applying a machine-learning model of the patient digital twin on the subject data and the experimental result data for the subject.

The subject data may indicate the subject has non-alcoholic fatty liver disease (NAFLD). The predictive model of the patient digital twin is trained on the subject data indicating the NAFLD. The biomimetic twin configuration data may indicate the patient biomimetic twin of the subject includes the NAFLD in the experimental representation of the patient biomimetic twin according to the subject data.

The testing configuration may indicate the subject NAFLD. The biomimetic twin configuration data indicates the patient biomimetic twin of the subject includes NAFLD in the experimental representation of the patient biomimetic twin according to the testing configuration.

The computer may apply the model on the subject data of a plurality of subjects. The computer outputs the patient digital twin as a generalized patient digital twin configured to generate a predicted outcome of the testing configuration generalized for the plurality of subjects.

The computer may iteratively train the patient digital twin using the subject data and the experimental result data for the particular subject, thereby outputting the patient digital twin as a customized patient digital twin tuned to generate a predicted outcome of the testing configuration for the particular subject.

The computer may identify a cohort of a plurality of subjects including the subject according to the testing configuration, by applying a cohort prediction engine of a machine-learning architecture on the subject data. The cohort prediction engine is trained to predict the plurality of subjects having a set of subject attributes relative to the testing configurations.

The computer may obtain, from a laboratory testing device, the experimental result data associated with a patient biomimetic twin of the subject.

The computer may generate a user interface presenting the patient biomimetic twin configuration of the biomimetic twin. The patient biomimetic twin configuration includes a set of subject attributes associated with the patient digital twin and the testing configuration.

The subject experimental representation of the patient biomimetic twin may be situated on a microfluidic chip. The subject experimental representation may include 3D layered cells housed in the microfluidic chip. The microfluidic chip may comprise one or more chambers comprising perfusion channels and injection ports. One or more cell types are introduced through the injection port or incorporated during assembly of the device. The one or more cell types may be selected from organoids, iPSC-derived cells, or primary cells. One or more of the microfluidic chips may be located in a perfusion module comprising a bio layer, a reservoir layer, and a gaseous pressure layer, supplying at least one of pneumatic or hydraulic pressure. One or more of the perfusion modules may be placed in a CO₂ cell incubator comprising an in-line imaging system.

In embodiments, a system includes one or more computers comprising at least processor and configured to perform certain operations or a computer-implemented method for developing and administering precision treatments. The computer may obtain subject data and a patient digital twin for a subject according to according to one or more treatment configurations indicating a treatment. The computer may obtain a predicted treatment outcome for the treatment according to a predictive model of the patient digital twin. The computer may construct a patient biomimetic twin for the subject based upon biomimetic twin configuration data indicated by the subject data for the subject. The computer may obtain experimental result data by subjecting the patient biomimetic twin to the treatment. The computer may enter the experimental result data into a computer, wherein the computer updates the predictive model of the patient digital twin based upon a level of error between the experimental result data and the predicted treatment outcome. The computer may administer the treatment to the subject.

The computer may iteratively update the patient digital twin using the subject data and a plurality of experimental result data iterations, thereby training the patient digital twin as a customized patient digital twin tuned to generate the predicted outcome for the treatment. The computer may select the one or more treatment configurations for developing a precision medicine protocol for the treatment. Iterations of the patient biomimetic twin are iteratively subjected to the treatment according to the one or more treatment configurations. The computer iteratively generates iterations of the predicted treatment outcome by iteratively applying the predictive model of the patient digital twin on the subject data in accordance with the one or more treatment configurations. The treatment may include the precision medicine protocol for treating non-alcoholic fatty liver disease (NAFLD). The subject data may indicate the subject has NAFLD. The predictive model of the patient digital twin is trained on the subject data indicating the NAFLD. The testing configuration may indicate the subject has NAFLD. The biomimetic twin configuration data indicates the patient biomimetic twin of the subject includes NAFLD in the experimental representation of the patient biomimetic twin according to the testing configuration.

The computer may execute a cohort prediction engine on the subject data of a plurality of subjects to predict a cohort of one or more subjects for the treatment. The computer may update the cohort prediction engine based upon the experimental result data obtained for each subject of the cohort. The predictive model of the patient digital twin may satisfy a threshold level of error prior to administering the treatment.

In embodiments, a system includes one or more computers comprising at least processor and configured to perform certain operations or a computer-implemented method for identifying subject cohorts. The computer may obtain subject data of a plurality of subjects, the subject data of each subject including one or more attributes of the subject. The computer may obtain a cohort of subjects for a treatment trial according to one or more treatment configurations, wherein a computer applies a cohort prediction engine on the subject data of the plurality of subjects using the one or more treatment configurations. For each subject of the cohort, the computer constructs a patient biomimetic twin for the subject based upon biomimetic twin configuration data indicated by the subject data for the subject. The computer may obtain the experimental result data associated with the patient biomimetic twin of the subject from a laboratory testing device by subjecting the patient biomimetic twin to the treatment for the treatment trial. The computer may administer the treatment to the cohort of subjects.

The computer may iteratively update the cohort prediction engine for iterative treatment trials. The computer iteratively updates the subjects for the cohort of subjects obtained for the treatment trial.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance may or may not occur, and that the description includes instances where said event or circumstance occurs and instances where it does not.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

In addition, where features or aspects of the disclosure are described in terms of Markush groups, those skilled in the art will recognize that the disclosure is also thereby described in terms of any individual member or subgroup of members of the Markush group.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

While various aspects and embodiments have been disclosed, other aspects and embodiments are contemplated. The various aspects and embodiments disclosed are for purposes of illustration and are not intended to be limiting, with the true scope and spirit being indicated by the following claims.

1. A computer-implemented method comprising:
obtaining, by a computer, subject data for a subject from one or more subject databases, the subject data including a plurality of types of subject data;
generating, by the computer, a predicted output by applying a predictive model of a patient digital twin on the subject data for the subject, the patient digital twin including the predictive model configured to generate a first predicted outcome of a testing configuration;
updating, by the computer, a parameter of the predictive model according to a user input;
generating, by the computer, a second predicted outcome by applying the patient digital twin on the subject data and the testing configuration, wherein the patient digital twin for the subject contains biomimetic twin configuration data associated with a patient biomimetic twin for the subject, and wherein the patient biomimetic twin includes an experimental representation of the subject according to the biomimetic twin configuration data;
obtaining, by the computer, experimental result data for the subject associated with the patient biomimetic twin for the subject and the testing configuration; and
updating, by the computer, the parameter of the patient digital twin, by applying a machine-learning model of the patient digital twin on the subject data and the experimental result data for the subject.

2. The method according to claim 1, wherein the subject data indicates the subject has non-alcoholic fatty liver disease (NAFLD), wherein the predictive model of the patient digital twin is trained on the subject data indicating the NAFLD. and wherein the biomimetic twin configuration data indicates the patient biomimetic twin of the subject includes the NAFLD in the experimental representation of the patient biomimetic twin according to the subject data.

3. The method according to claim 1, wherein the testing configuration indicates the subject has NAFLD, and wherein the biomimetic twin configuration data indicates the patient biomimetic twin of the subject includes NAFLD in the experimental representation of the patient biomimetic twin according to the testing configuration.

4. The method according to claim 1, wherein the computer applies the model on the subject data of a plurality of subjects, and wherein the computer outputs the patient digital twin as a generalized patient digital twin configured to generate a predicted outcome of the testing configuration generalized for the plurality of subjects.

5. The method according to claim 1, wherein the computer iteratively trains the patient digital twin using the subject data and the experimental result data for the particular subject, thereby outputting the patient digital twin as a customized patient digital twin tuned to generate a predicted outcome of the testing configuration for the particular subject.

6. The method according to claim 1, further comprising identifying, by the computer, a cohort of a plurality of subjects including the subject according to the testing configuration, by applying a cohort prediction engine of a machine-learning architecture on the subject data, the cohort prediction engine trained to predict the plurality of subjects having a set of subject attributes relative to the testing configurations.

7. The method according to claim 1, wherein obtaining the experimental result data includes receiving, by the computer from a laboratory testing device, the experimental result data associated with the patient biomimetic twin of the subject.

8. The method according to claim 1, further comprising generating, by the computer, a user interface presenting the patient biomimetic twin configuration of the biomimetic twin, the patient biomimetic twin configuration including a set of subject attributes associated with the patient digital twin and the testing configuration.

9. The computer-implemented method of claim 1, wherein the subject experimental representation of the patient biomimetic twin is situated on a microfluidic chip.

10. The computer-implemented method of claim 9, wherein the subject experimental representation includes 3D layered cells housed in the microfluidic chip.

11. The computer-implemented method of claim 9, wherein the microfluidic chip comprises one or more chambers comprising perfusion channels and injection ports.

12. The computer-implemented method of claim 11, wherein one or more cell types are introduced through the injection port or incorporated during the assembly of the device, and wherein the one or more cell types are selected from organoids, iPSC-derived cells, or primary cells.

13. The computer-implemented method of claim 9, wherein one or more of the microfluidic chips are located in a perfusion module comprising a bio layer, a reservoir layer, and a gaseous pressure layer, supplying at least one of pneumatic or hydraulic pressure.

14. The computer-implemented method of claim 13, wherein one or more of the perfusion modules are placed in a CO₂ cell incubator comprising an in-line imaging system.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method for developing and administering precision treatments comprising:
obtaining subject data and a patient digital twin for a subject according to according to one or more treatment configurations indicating a treatment;
obtaining a predicted treatment outcome for the treatment according to a predictive model of the patient digital twin;
constructing a patient biomimetic twin for the subject based upon biomimetic twin configuration data indicated by the subject data for the subject;
obtaining experimental result data by subjecting the patient biomimetic twin to the treatment;
entering the experimental result data into a computer, wherein the computer updates the predictive model of the patient digital twin based upon a level of error between the experimental result data and the predicted treatment outcome; and
administering the treatment to the subject.

28. The method according to claim 27, wherein the computer iteratively updates the patient digital twin using the subject data and a plurality of experimental result data iterations, thereby training the patient digital twin as a customized patient digital twin tuned to generate the predicted outcome for the treatment.

29. The method according to claim 28, further comprising selecting the one or more treatment configurations for developing a precision medicine protocol for the treatment,
wherein iterations of the patient biomimetic twin are iteratively subjected to the treatment according to the one or more treatment configurations, and
wherein the computer iteratively generates iterations of the predicted treatment outcome by iteratively applying the predictive model of the patient digital twin on the subject data in accordance with the one or more treatment configurations.

30. The method according to claim 29, wherein the treatment includes the precision medicine protocol for treating non-alcoholic fatty liver disease (NAFLD).

31. The method according to claim 29, wherein the subject data indicates the subject has non-NAFLD, and wherein the predictive model of the patient digital twin is trained on the subject data indicating the NAFLD.

32. The method according to claim 29, wherein the testing configuration indicates the subject NAFLD, and wherein the biomimetic twin configuration data indicates the patient biomimetic twin of the subject includes NAFLD in the experimental representation of the patient biomimetic twin according to the testing configuration.

33. The method according to claim 27, wherein the computer executes a cohort prediction engine on the subject data of a plurality of subjects to predict a cohort of one or more subjects for the treatment.

34. The method according to claim 33, wherein the computer updates the cohort prediction engine based upon the experimental result data obtained for each subject of the cohort.

35. The method according to claim 27, wherein the predictive model of the patient digital twin has satisfied a threshold level of error prior to administering the treatment.

36. A method for identifying subject cohorts comprising:
obtaining subject data of a plurality of subjects, the subject data of each subject including one or more attributes of the subject;
obtaining a cohort of subjects for a treatment trial according to one or more treatment configurations, wherein a computer applies a cohort prediction engine on the subject data of the plurality of subjects using the one or more treatment configurations;
for each subject of the cohort, constructing a patient biomimetic twin for the subject based upon biomimetic twin configuration data indicated by the subject data for the subject;
obtaining the experimental result data associated with the patient biomimetic twin of the subject from a laboratory testing device by subjecting the patient biomimetic twin to the treatment for the treatment trial; and
administering the treatment to the cohort of subjects.

37. The method according to claim 36, wherein the computer iteratively updates the cohort prediction engine for iterative treatment trials, and wherein the computer iteratively updates the subjects for the cohort of subjects obtained for the treatment trial.
